(19) United States
(12) Patent Application Publication
(10) Pub. No.: US 2024/0099306 A1
(43) Pub. Date: Mar. 28, 2024

(54) Antimicrobial Component for an Aerosol Generating System

(71) Applicant: JT International SA, Geneva (CH)

(72) Inventor: Akira Yamaguchi, Geneva (CH)

(73) Assignee: JT International SA, Geneva (CH)

(21) Appl. No.: 18/038,142

(22) PCT Filed: Dec. 13, 2021
(86) PCT No.: PCT/EP2021/085555

§ 371 Date: May 22, 2023

(30) Foreign Application Priority Data
Dec. 15, 2020 (EP) 20214167.7

(51) Int. Cl. A01N 59/20; A01N 25/28; A24F 40/42; A24F 40/85; B82Y 5/00

(52) U.S. Cl. CPC A01N 59/20; A01N 25/28; A24F 40/42; A24F 40/85; B82Y 5/00

(57) ABSTRACT

A component is provided for an aerosol generating system; wherein at least a portion of the exterior of the component includes antimicrobial particles; and wherein the antimicrobial particles include monovalent copper compound particles. By providing the component in this manner, the likelihood of harmful bacteria or viruses being transmitted via the component is reduced.

### BACKGROUND

Aerosol generating devices have become popular alternatives to traditional combustible tobacco products. Heated tobacco products, also referred to as heat-not-burn products are one class of aerosol generating device that are configured to heat a tobacco substrate to a temperature that is sufficient to produce an aerosol from the substrate but is not so high that the tobacco combusts. Although this specification makes reference to heated tobacco products in particular, it will be appreciated that the discussion that follows applies equally to aerosol generating systems that incorporate other kinds of heatable substrate.

Users of aerosol generating devices may often face situations where they hold their devices after they have touched surfaces that may be infected with harmful germs, bacteria and/or viruses such as a door handle when entering or exiting a space or in public transportations when holding a handrail on a door or a seat, or in the access passage of a vehicle, or in the train, or in the autobus. Another situation is for example when one person carrying the germs, bacteria or virus in his hands, carries the aerosol generating device for another person. In these situations, users are concerned about the risk of contamination or transmitting harmful bacteria or viruses through the systems, particularly as aerosol generating devices which are often in close contact with the hands and mouth of users.

Existing attempts to reduce the risk of transmission of such systems include cleaning the device between uses by different users, or replacing/uncovering a system component such as a mouthpiece before using the system. However, such techniques are often inconvenient for the users, susceptible to human error, or lack sufficient antimicrobial effectiveness.

Accordingly, there is a need for an aerosol generating system that addresses these issues.

### SUMMARY

According to a first aspect of the invention, there is provided a component for an aerosol generating system; wherein at least a portion of the exterior of the component comprises antimicrobial particles; and wherein the antimicrobial particles comprises monovalent copper compound particles.

It has been found that monovalent copper compound particles may effectively reduce the activity of microorganisms including bacteria and viruses. As a portion of the exterior of a provided component for an aerosol generating system comprises these particles, the component itself also holds the desirable antimicrobial properties. In this way, the likelihood of harmful bacteria or viruses surviving on the component and/or being transmitted via the component is reduced. This increases the safety of such components as well as the convenience of their use.

Typically, it is the exterior of a component for an aerosol generating system that is handled most frequently by a user and so is most likely to pick up harmful bacteria or viruses. Therefore, including the monovalent copper compound particles in, at least a portion of, the exterior of the component ensures the antimicrobial effects are provided where they are most needed without unnecessarily incorporating the monovalent copper compound particles throughout the whole component. Including the monovalent copper compound particles in areas of the component that are unlikely to be contacted by users, such as on some interior components or hidden surfaces, may be an ineffective use of the particles and waste resources.

While much of this disclosure focusses on monovalent copper compound particles, other antimicrobial particles may also be included in the portion of the exterior of the component if the antimicrobial properties provided supplement the effects of the monovalent copper compound particles.

Preferably, the monovalent copper compound particles comprise monovalent copper compound nanoparticles. In this way, the performance of antimicrobial effects is increased relative to larger sized particles, as the small size of the monovalent copper compound nanoparticles allows for a greater quantity of particles to be comprised in the portion, and the nanoparticles have a higher surface area to volume ratio than larger monovalent copper compound particles. Preferably, the diameter of the monovalent copper compound particles is between 80 nm and 120 nm. More preferably, the diameter of the monovalent copper compound particles is between 90 nm and 110 nm. Most preferably, the diameter of the monovalent copper compound particles is 100 nm.

The antimicrobial particles may be arranged throughout the portion. In this way, portion itself comprises the monovalent copper compound particles and so will retain the associated antimicrobial properties even if the exterior of the portion is scratched or damaged (e.g. if the component is dropped by a user).

The portion of the exterior of the component may comprise a coating where the antimicrobial particles are arranged throughout the coating. Adding an antimicrobial coating to the exterior of the device allows antimicrobial properties to be added to a portion that did not previously have them, or enhances the antimicrobial properties already present in a portion (e.g. if the portion comprises monovalent copper compound particles arranged throughout the portion). Furthermore, the concentration of antimicrobial particles in a coating can be easily adjusted to provide a high level of control over the antimicrobial properties of the component during or after component fabrication. Multiple layers of the antimicrobial coating may be applied to the portion of the exterior of the coating to increase the antimicrobial effects of the component and to increase resistance to loss of these effects if the exterior of the portion is scratched or damaged.

The portion of the exterior of the component may be configured to be held by a user. These portions are likely to be handled and touched more frequently than other areas of the component, therefore it is beneficial that portions configured to be held by the user comprise the monovalent copper compound particles. For example, when the component for an aerosol generating system is an aerosol generating device, the portion of the exterior of the component may comprise a grip for the device. In this example, the grip comprises the monovalent copper compound particles and so the microorganism activity on the grip is reduced. As the microorganism activity on the grip (a portion of the component configured and likely to be held by a user) is reduced, the risk of bacteria or viruses being transmitted from the grip to the user is reduced.

The portion of the exterior of the component may comprise a first area and a second area; wherein the concentration of antimicrobial particles in the first area than the concentration of antimicrobial particles in the second area.

In this way, the antimicrobial properties provided by the antimicrobial particles may be adjusted in different areas of the component. Preferably, the concentration or density of antibacterial particles in a given area or portion is determined at least partially based on the area placement and use (e.g. how frequently the area is interacted with by a user, and how the interaction occurs). For example, a first area of the portion may be configured to be held or touched more frequently than a second area of the portion (though this second area is still likely to be contacted by a user) and so the first area may comprise a higher concentration of monovalent copper compound particles than the second area; this saves costs and resources while still ensuring the safety of the component (by including particles in both areas of the portion). In another example, a first area of the portion may be a mouthpiece of a component that is configured to be brought into contact with a user's mouth and therefore strong antimicrobial properties (provided by a high concentration of monovalent copper compound particles) are desirable in this first area.

The component may be an aerosol generating device. Such devices are regularly contacted by the hands and mouth of users so it is desirable to reduce the activity of harmful microorganisms on these devices. In addition, aerosol generating devices may often be held by several different people so it is beneficial to these users that the antimicrobial properties reduce the chance of harmful viruses and bacteria being transmitted between different users.

The component may be an accessory for the aerosol generating system. Accessories for aerosol generating systems include, but are not limited to, those used to contain, protect and carry other components in the aerosol generating system. For example, a carry case for an aerosol generating system may be used to store and transport an aerosol generating device and spare cartridges for the device when not in use. Providing such a carry case with monovalent copper compound particles in its exterior helps prevent harmful bacteria or viruses from being transmitted via the carry case.

The exterior of the component may comprise a textile material. Specifically, the portion of the exterior of the component may comprise a textile material. Though many components of aerosol generating systems comprise metals or polymers, some components (such as a fabric carry case or a grip for an aerosol generating device) comprise textile materials and it is important that the antimicrobial properties of the monovalent copper compound particles may still be provided in these components.

The component may be a cartridge for an aerosol generating device. These cartridges often comprise a mouthpiece, an aerosolisable substrate, or both. Such cartridges are typically inserted and removed from an aerosol generating device by hand and so it is desirable that they are provided with antimicrobial properties, so as to reduce the likelihood of transmission via the cartridge.

The exterior of the component may comprise a polymer. Specifically, the portion of the exterior of the component may comprise a polymer. A large amount of components for aerosol generating systems comprise polymer materials and/or metal materials, so it is desirable that the antimicrobial properties of monovalent copper compound particles are present in these materials.

According to a second aspect of the invention, there is provided a method for producing a component according to any example of the first aspect, the method comprising: covering at least a portion of the exterior of the component with a coating; wherein the coating comprises antimicrobial particles, and the antimicrobial particles comprise monovalent copper compound particles.

In this way, antimicrobial properties may be introduced to a component for an aerosol generating system before or after the component has been manufactured. For example, if the component is an aerosol generating device with then an outer housing of the device may be covered with this coating as part of the device manufacturing process; alternatively, the device may have already been bought and used by a user for a period of time before the coating is applied to a portion of the exterior of the device. It should be noted that multiple layers of the coating may be applied to the same portion of the exterior of the component. Furthermore, separate coatings with different concentrations of antimicrobial particles may be provided to fine tune the antimicrobial properties of the component.

The method may further comprise, before covering the portion, dispersing the antimicrobial particles through a coating fluid to form the coating. In this way, the antimicrobial particles, such as monovalent copper compound particles, can easily be evenly dispersed through the coating fluid before the coating is applied to the component exterior. This enables accurate control of the provided antimicrobial properties and allows coating mixtures (with known concentrations of antimicrobial particles) to be pre-made before application.

According to a third aspect of the invention, there is provided a method for producing a component for an aerosol generating system, the method comprising: providing a component portion material; dispersing antimicrobial particles throughout the component portion material to form an antimicrobial material, wherein the antimicrobial particles comprise monovalent copper compound particles; and forming at least a portion of the component from the antimicrobial material.

As the antimicrobial particles (including the monovalent copper compound particles) are arranged within the component itself, the antimicrobial properties provided by the particles will be retained even if the exterior of the portion of the component is damaged (e.g. if the component is scratched or dropped). The remainder of the component may be produced in the conventional manner for the given type of component.

If the component being produced comprises a plurality of portions that comprise antimicrobial particles, in some examples of the invention different portions may comprise different concentrations of antimicrobial particles. In these examples, the concentration of antimicrobial particles may be adjusted by changing the ratio of the component portion material to the number of antimicrobial particles prior to the forming step, and preferably during the dispersing step or the providing step.

The component portion material may comprise a textile material, and the step of dispersing antimicrobial particles comprises: arranging antimicrobial particles through the fibre network of the textile material.

The component material may comprise a polymer, and the step of dispersing antimicrobial particles comprises: dispersing the antimicrobial particles throughout the polymer.

The step of dispersing the antimicrobial particles throughout the polymer may comprise dispersing the antimicrobial particles throughout the polymer when the polymer is a fluid.

This facilitates an even dispersion of the antimicrobial particles throughout the polymer. In many cases where the component material comprises a polymer, the polymer is a thermoplastic or thermosetting polymer that is formed into the component through processes such as injection moulding or blow moulding while the polymer is in a fluid (or molten) state. In these examples, it is not necessary to return the polymer to a solid state after dispersing the antimicrobial particles and so this may save time and resources during the component manufacturing process.

In this disclosure, a component for an aerosol generating system refers to a constituent device, apparatus, or part of a device or apparatus that makes up an aerosol generating system. For example, an aerosol generating system may comprise an aerosol generating device, a cartridge (for use with an aerosol generating device), an accessory for an aerosol generating system (such as a carry case for an aerosol generating device), or a combination thereof. It will be appreciated that aspects of the invention may also be applied to other components for an aerosol generating system not explicitly named above.

### BRIEF DESCRIPTION OF THE DRAWINGS

Aspects of the invention will now be described, by way of example only, with reference to the accompanying drawings, in which:

FIG. 1A schematically illustrates an example aerosol generating device;

FIG. 1B schematically illustrates another example aerosol generating device;

FIG. 1C schematically illustrates an example cartridge for an aerosol generating system;

FIG. 1D schematically illustrates an example accessory for an aerosol generating system;

FIG. 2 schematically illustrates an example portion of a component for an aerosol generating system;

FIG. 3 schematically illustrates another example portion of a component for an aerosol generating system, where the portion comprises a coating; and

FIG. 4 schematically illustrates a further example portion of a component for an aerosol generating system, where the portion comprises a textile material.

### DETAILED DESCRIPTION

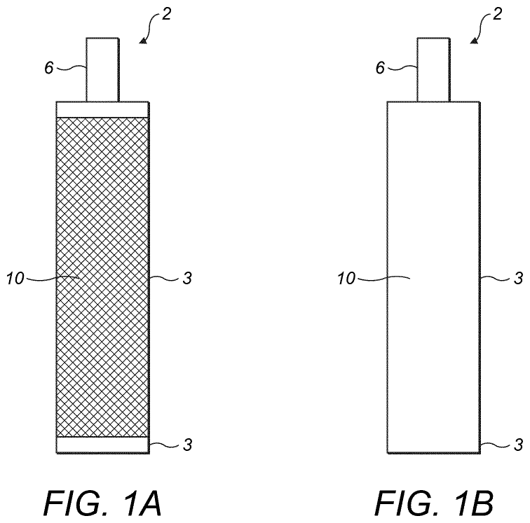

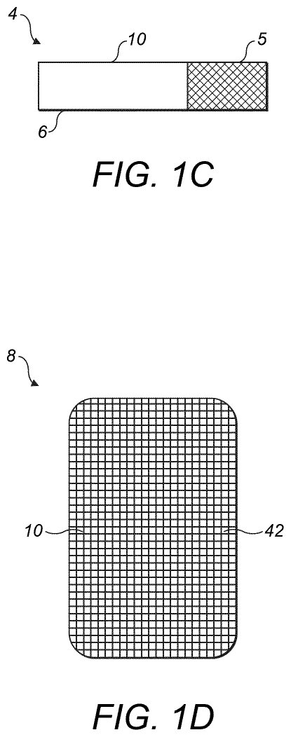

The present invention discloses components for aerosol generating systems, where the components have antimicrobial properties provided by antimicrobial particles 20 distributed through a portion 10 of the exterior of a component. FIGS. 1A to 1D show several examples of such components and FIGS. 2, 3, and 4 show examples of how the antimicrobial particles 20 may be comprised within the component portion 10.

Specifically, the antimicrobial particles 20 discussed herein comprise monovalent copper compound particles 22. The monovalent copper compound particles 22 may reduce microorganism activity and improve hygiene through several mechanisms; one such mechanism is described below. When the monovalent copper compound particles 22 contact microorganisms such as bacteria or viruses, the monovalent copper compound particles 22 generate monovalent copper ions (Cu⁺). These copper ions react with oxygen (O₂) in the surrounding air and generate reactive oxygen species such as hydroxide ions (OH⁻). Further monovalent copper ions act together with the hydroxide ions with antimicrobial effects against the microorganisms. An antimicrobial effect or property refers to the effect of eliminating, rendering inactive, or halting the reproduction/growth of microorganisms.

An advantage of using monovalent copper compound particles 22 rather than other copper particles is that the monovalent copper ions are oxidised more readily than bivalent copper ions and so are more effective at reducing microorganism activity. Monovalent copper compound particles 22 have been found to reduce bacterial and viral activity in a sample by over 99.9% in less than an hour.

In some examples of the invention, the monovalent copper compound particles 22 comprise monovalent copper compound nanoparticles. The performance of antimicrobial effects provided by these nanoparticles is greater than such effects provided by larger sized particles of the same material. This is due at least in part to the higher surface area to volume ratio of the nanoparticles and the smaller size allowing for a greater number of monovalent copper compound particles 22 to be distributed through the portion 10.

An example aerosol generating device 2 is generally illustrated in FIG. 1A. The device 2 comprises an exterior housing 3 that holds interior components (not shown) of the device 2 used for generation of an aerosol. For example, these interior components may include an aerosol generating substrate disposed within a heating chamber. In use, the substrate is heated by a nearby heater, powered by a battery, to generate an aerosol for subsequent inhalation by a user.

The device 2 further comprises a mouthpiece 6 for a user to inhale an aerosol through. In this example the mouthpiece 6 is a part of the aerosol generating device, while in other examples it may be a separate component or a part of a separate component such as a cartridge.

In the example of FIG. 1A, a portion 10 of the aerosol generating device 2 has antimicrobial properties. These antimicrobial properties are provided by antimicrobial particles 20 comprised in the portion 10 of the exterior housing 3, these particles 20 are described in more detail below. As shown in FIG. 1A, the portion 10 covers regions of the device 2 that are frequently held by a user; thereby reducing the likelihood of harmful bacteria or viruses being transmitted between separate users through sharing of the device 2.

In some examples of the invention, the exterior housing 3 comprises a grip configured to be held by a user, and in some other examples the device 2 comprises a grip that is distinct from the housing 3 (e.g. a layer of textile material wrapped around a portion of the exterior housing 3). In both examples, it is preferred that the grip comprises the antimicrobial particles 20.

FIG. 1B shows another example of an aerosol generating device 2. In contrast with the device 2 of FIG. 1A, the whole of the exterior of the device 2 comprises the antimicrobial particles 20. This increases the antimicrobial properties of the device 2, reducing the risk of harmful bacteria or viruses being transmitted irrespective of where or how the device 2 is handled by a user.

In FIG. 1A, the portion 10 is shaded to clearly distinguish the portion 10 comprising antimicrobial particles 20 from other portions of the device 2 that do not comprise antimicrobial particles 20. In FIG. 1B, the whole exterior of the device 2 comprises antimicrobial particles 20; therefore, the shading has not been included as it is not necessary to distinguish between portions of the component comprising the particles 20 and portions not comprising the particles 20.

In some examples of the invention, the concentration or density of antimicrobial particles 20 may vary across different portions 10 of a component or areas of a portion 10 of a component. This allows the antimicrobial properties provided by the particles 20 to be adjusted across the component; saving resources and reducing component costs while still reducing the activity of harmful microorganisms on the component. For example, where the component is an aerosol generating device similar to that in FIGS. 1A and 1B, the mouthpiece and grip portions of the device may comprise a higher concentration of antimicrobial particles 20 than other areas of the device. The mouthpiece and grip portions are likely to be handled more frequently by a user and so the most effective antimicrobial effects are required here.

FIG. 1C shows a cartridge 4 for an aerosol generating system. The cartridge 4 comprises an aerosolisable substrate 5 and mouthpiece 6 and is configured to be at least partially received within an aerosol generating device. As cartridges for aerosol generating systems are typically physically handled by a user, e.g. inserted and removed from a device by hand, it is advantageous that at least a portion of the exterior of such a cartridge 4 comprises antimicrobial particles 20 to provide antimicrobial effects.

In the cartridge 4 of FIG. 1C, the portion of the exterior of the cartridge 4 around the mouthpiece 6 comprises the antimicrobial particles 20. In this example, antimicrobial particles 20 are not provided on the portion of the exterior of the cartridge 4 at the substrate 5 as, in use, this portion of the cartridge 4 is heated to high temperatures that reduce the activity of many harmful bacteria and viruses; and so the risk of these microorganisms being transmitted is lower. In addition, users typically avoid touching the substrate 5 of the cartridge 4 in order to prevent residue from the substrate 5 being deposited on their person. However, in some examples of the invention the portion of the cartridge 4 at the substrate 5 may comprise the antimicrobial particles 20 in order to further increase the safety of the component.

While the cartridge 4 in the example of FIG. 1C comprises both a substrate 5 and a mouthpiece 6, in other examples the cartridge 4 may comprise different parts. For example, the cartridge 4 may comprise an aerosol cooling region arranged between the mouthpiece 6 and substrate 5, while in another example the cartridge 4 may not include the mouthpiece 6 and only comprises the substrate 5. In such components, the distribution of antimicrobial particles 20 are distributed according to the risk associated with each portion the component.

FIG. 1D shows an example accessory 8 for an aerosol generating system. Specifically, the accessory 8 is a fabric carry case comprised of a textile material and used to store other elements of an aerosol generating system, such as a cartridge 4 and aerosol generating device 2, when they are not in use. In the example of FIG. 1D, the whole of the exterior of the accessory 8 comprises the antimicrobial particles 20 to maximise the antimicrobial properties of the carry case.

FIG. 2 schematically illustrates a portion 10 of the exterior of a component for an aerosol generating system with antimicrobial particles 20 arranged throughout the portion 10. Preferably, the particles 20 are evenly distributed to ensure antimicrobial effects are provided across the full surface area of the portion 10. If the component exterior is a plastic material, polymeric material, or metal material then the antimicrobial particles 20 are typically arranged through the component in this manner.

A portion 10 of a component as shown in FIG. 2 may be produced by first providing a component portion material 40—that is, the main structural material used to form a portion of the exterior of the component. Next, antimicrobial particles 20 are dispersed throughout the component portion material 40 to form an antimicrobial material 42. Subsequently, a portion 10 of the component is formed from the antimicrobial material 42; retaining the structural properties provided by the component portion material 40 while also gaining the antimicrobial benefits of the antimicrobial particles 20.

The precise nature of the dispersing step and the forming step will vary depending on the properties of the component portion material 40. For example, when the component portion material 40 comprises a polymer (e.g. for forming the exterior housing of an aerosol generating device), then it is preferable to disperse the antimicrobial particles 20 throughout the polymer when it is in a fluid or melt phase.

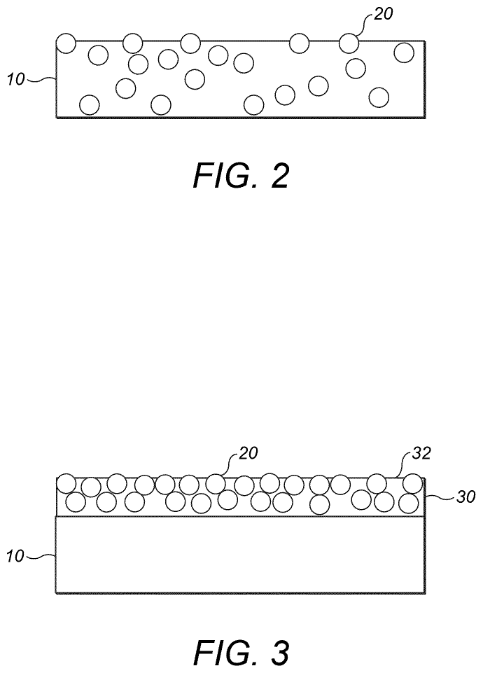

FIG. 3 shows another portion 10 of the exterior of a component for an aerosol generating system. The portion 10 comprises a coating 30 that covers the component exterior and comprises antimicrobial particles 20 arranged throughout the coating 30. Typically, the coating 30 comprises a coating fluid 32 and the antimicrobial particles 20 are dispersed throughout the coating fluid 32 before the coating 30 is applied to the portion 10. This helps provide an even dispersion of the particles 20 throughout the coating 30 and allows the concentration of antimicrobial particles 20 within the coating 30 to be selected prior to application. Applying an antimicrobial coating 30 to a portion of a component may be cheaper than dispersing antimicrobial particles 20 throughout the component portion material 40 itself. In addition, the coating 30 may be easily applied to existing components (to either completed components or at the end of a component manufacturing process) and provide them with antimicrobial properties.

The coating 30 may be applied to the portion 10 of the component before or after the component is assembled. For example, if the portion 10 is a grip for an aerosol generating device then the coating 30 can be applied to the grip before it is connected to the device or after it is connected to the device.

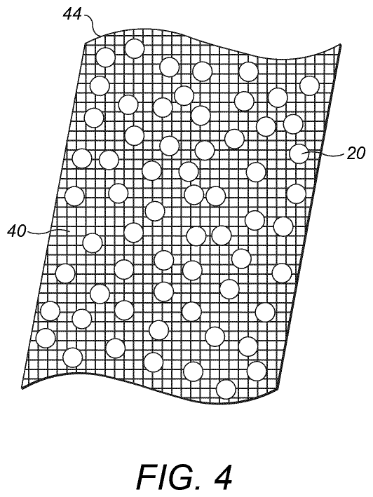

The portion 10, of the exterior of a component for an aerosol generating system, shown in FIG. 4 comprises a textile material 44. This may be, for example, a portion 10 of the carry case accessory shown in FIG. 1D. The textile material 44 is made up from a network of interlacing fibres, referred to as a fibre network 46. As shown in FIG. 4, the antimicrobial particles 20 are arranged throughout the fibre network 46. Preferably, the particles 20 are dispersed through the fibres before the network 46 is constructed and before the portion 10 of the component is formed from the textile material 44. This provides a textile material 44 that is an antimicrobial material 42 and so an even distribution of antimicrobial particles 20 through the resulting portion 10.

The coating 30 shown in FIG. 3 may also be applied to portions 10 that have antimicrobial particles 20 arranged throughout the portion 10, like those shown in FIGS. 2 and 4. This may increase the antimicrobial properties of the portion 10 and provides the portion 10 with two layers of antimicrobial protection. For instance, if the outer layer (the coating 30) is scratched or damaged then the portion retains the benefits of the antimicrobial particles 20 due to their dispersion throughout the portion 10 underneath in the second layer.

1-14. (canceled)

15. A component for an aerosol generating system;
wherein at least a portion of an exterior of the component comprises antimicrobial particles;
wherein the antimicrobial particles comprise monovalent copper compound particles.

16. The component according to claim 15, wherein the monovalent copper compound particles comprise monovalent copper compound nanoparticles.

17. The component according to claim 15, wherein the antimicrobial particles are arranged throughout the portion.

18. The component according to claim 15, wherein the portion comprises a coating; and
the antimicrobial particles are arranged throughout the coating.

19. The component according to claim 15, wherein the portion is configured to be held by a user.

20. The component according to claim 15, wherein the portion of the exterior of the component comprises a first area and a second area; and a concentration of the antimicrobial particles in the first area is greater than a concentration of the antimicrobial particles in the second area.

21. The component according to claim 15, wherein the component is an aerosol generating device.

22. The component according to claim 15, wherein the component is an accessory for the aerosol generating system.

23. The component according to claim 15, wherein the component is a cartridge for an aerosol generating device.

24. The component according to claim 15, wherein the exterior of the component comprises a polymer.

25. A method for producing a component for an aerosol generating system, the method comprising:
covering at least a portion of an exterior of the component with a coating;
wherein the coating comprises antimicrobial particles, and the antimicrobial particles comprise monovalent copper compound particles.

26. The method according to claim 25, wherein before the step of covering, the method further comprises:
dispersing the antimicrobial particles through a coating fluid to form the coating.

27. A method for producing a component for an aerosol generating system, the method comprising:
providing a component portion material;
dispersing antimicrobial particles throughout the component portion material to form an antimicrobial material, wherein the antimicrobial particles comprise monovalent copper compound particles; and
forming at least a portion of the component from the antimicrobial material.

28. The method according to claim 27, wherein the component portion material comprises a textile material, and the step of dispersing the antimicrobial particles comprises:
arranging the antimicrobial particles throughout a fibre network of the textile material.

29. The method according to claim 27, wherein the component portion material comprises a polymer, and the step of dispersing the antimicrobial particles comprises:
dispersing the antimicrobial particles throughout the polymer.
